(19) United States
(12) Patent Application Publication
(10) Pub. No.: US 2022/0296780 A1
(43) Pub. Date: Sep. 22, 2022

(54) BIOCERAMIC-CONTAINING THERMOPLASTIC EXTRUSION AND METHOD OF SURGICAL IMPLANT MANUFACTURE

(71) Applicant: OrthoMod LLC, Dayton, OH (US)

(72) Inventors: David Louis Kirschman, Dayton, OH (US); Dewey J. Weeda, Oakwood, OH (US); Shawn A. Hunter, Springboro, OH (US)

(21) Appl. No.: 17/655,148

(22) Filed: Mar. 16, 2022

Related U.S. Application Data
(60) Provisional application No. 63/161,563, filed on Mar. 16, 2021.

(51) Int. Cl. A61L 27/46; A61L 27/56; A61L 27/36

(52) U.S. Cl. CPC A61L 27/46; A61L 27/56; A61L 27/365; A61L 2430/02

(57) ABSTRACT

A method of generating a bioceramic-containing biomaterial-derived thermoplastic extrusion is provided. The method includes combining a bioceramic-containing solid with at least one thermoplastic resin, wherein the bioceramic-containing solid is uniformly dispersed in the resin. The method further includes extruding the bioceramic-containing solid included in the resin to create a net shape. The net shape is selected from a group consisting of a filament, a pellet, a bar, a molding, and a three-dimensional printing material stock.

### CROSS REFERENCE TO RELATED APPLICATIONS

The present application claims the filing benefit of U.S. Provisional Application No. 63/161,563, filed on Mar. 16, 2021, the disclosure of which is incorporated herein by reference in its entirety.

### BACKGROUND

Human-derived bone grafts are commonly used in the treatment of orthopedic pathologies and injuries. Such grafts have the benefits of consolidating into host bone and promoting healing through bony fusion or arthrodesis. However, there are significant limitations to the application of natural bone allografts or xenografts to such treatments. Natural bone is available in limited anatomical shapes and sizes that may not be adequate for treatment of certain orthopedic pathologies. The ability to machine or form bone is limited for similar reasons. Recently, there have been advances in the use of three dimensional or volumetric methods for the manufacture of complex or customized medical devices. The purpose of this invention is to practically combine a bioceramic-containing component into a thermoplastic filament that can be used for the manufacture of medical devices having both mechanical and biological function in myriad shapes and forms. What is needed is an improved system, method, and processes for manufacturing an implant that has improved osteoconductive capabilities and/or provides improved means for manufacturing an implant and selective placement of bone therein to promote osteoconduction without using human-derived or animal-derived bone grafts.

### SUMMARY

Described herein are methods and systems related to artificially-derived bone grafts for implantation in humans. Particularly, in an embodiment, A method of generating a bioceramic-containing biomaterial-derived thermoplastic extrusion is provided. the method includes combining a bioceramic-containing solid with at least one thermoplastic resin, wherein the bioceramic-containing solid is uniformly dispersed in the resin. The method further includes extruding the combined bioceramic-containing solid and the at least one thermoplastic resin to form an extrusion and to create a net shape. The net shape may be selected from a group consisting of a filament, a pellet, a bar, a molding, and a three-dimensional printing material stock.

In a related embodiment, mixing the bioceramic-containing solid with a thermoplastic pellet in a solid state occurs prior to or during extruding the bioceramic-containing solid and the at least one thermoplastic resin. Mixing the bioceramic-containing solid with the thermoplastic pellet occurs below a glass transition temperature of the thermoplastic pellet, and the mixing further includes physical agitation, electrostatic adhesion, or ultrasonic agitation to create uniform mixing of the bioceramic-containing solid and the thermoplastic resin.

In a related embodiment, mixing the bioceramic-containing solid with the at least one thermoplastic resin occurs within an extrusion chamber subjected to heat and/or pressure by an auger screw. The auger screw is configured to disperse the bioceramic-containing solid in the at least one thermoplastic resin.

In a related embodiment, the bioceramic-containing solid is mixed with the at least one thermoplastic resin in a liquid state by undergoing mechanical agitation prior to or during the extrusion process.

In a related embodiment, the method further includes mixing the bioceramic-containing solid with a thermoplastic liquid to create a uniform dispersal prior to being placed in an extrusion chamber. The mixing includes impeller agitation or ultrasonic agitation resulting in a heated liquid state, the mixed bioceramic-containing solid and thermoplastic liquid having a temperature above the melting point of the thermoplastic liquid, wherein the bioceramic-containing solid is added during and/or prior to the agitation and/or heating.

In a related embodiment, the bioceramic-containing solid includes at least one of calcium phosphate, tricalcium phosphate, hydroxyapatite, multiphasic calcium phosphate, calcium silicate, sodium silicate, or silicate-substituted calcium phosphate.

In a related embodiment, the bioceramic-containing solid is provided in a powdered or granular form having particles equal to or less than 500 μm in size.

In a related embodiment, the bioceramic-containing solid is mixed with the thermoplastic resin in a predetermined ratio, the ratio is determined by mass, wherein the mass of the thermoplastic resin is from 10 to 50 times the mass of the bioceramic-containing solid.

In a related embodiment, the filament is configured to roll onto a spool.

In a related embodiment, the extrusion undergoes terminal sterilization via an irradiation, heat, or chemical treatment.

In another embodiment, a bioceramic-containing biomaterial-derived thermoplastic extrusion is provided. The bioceramic-containing biomaterial-derived thermoplastic extrusion includes a solid derived from bioceramic-containing biomaterial, the bioceramic-containing biomaterial is uniformly dispersed in a thermoplastic resin, and the bioceramic-containing biomaterial-derived thermoplastic extrusion is shaped as a filament, pellet, bar, molding, or three-dimensional printing material.

In a related embodiment, the bioceramic-containing biomaterial includes at least one of calcium phosphate, tricalcium phosphate, hydroxyapatite, multiphasic calcium phosphate, calcium silicate, sodium silicate, or silicate-substituted calcium phosphate.

In a related embodiment, the thermoplastic resin comprises nylon, ABS, polycarbonate, acrylic, polyaryletherketones, polymethyl methacrylate, polycaprolactone, or polyetherimide.

In a related embodiment, the a bioceramic-containing biomaterial-derived thermoplastic extrusion further includes a minimum of 0.1% bioceramic-containing biomaterial by weight.

In a related embodiment, the extrusion is formed into a filament, the filament being substantially flexible, such that the filament is configured to be rolled onto a spool.

In a related embodiment, the extrusion undergoes terminal sterilization via an irradiation, heat, or chemical treatment.

In another embodiment, an osteoconductive surgical implant is provided. The osteoconductive surgical implant includes a bioceramic-containing biomaterial-derived thermoplastic extrusion, wherein the surgical implant incorporates a combination of a bioceramic-containing solid and a thermoplastic with dispersal of the bioceramic-containing solid in the thermoplastic.

In a related embodiment, the osteoconductive surgical implant is manufactured utilizing additive manufacturing, volumetric printing, injection molding, machining, sintering, or forming.

In a related embodiment, the dispersal of the bioceramic-containing solid within the thermoplastic is uniform.

In a related embodiment, at least a portion of the bioceramic-containing solid is exposed at a surface of the implant, and the exposed bioceramic-containing solid expresses osteoconductive properties and imparting the properties to the implant.

In a related embodiment, the bioceramic-containing solid is mechanically or chemically exposed on the surface in a controlled manner for exposure of osteoconductive elements where biologic response is desired, wherein the chemical exposure includes treatment of the implant with an acid, ethanol, or a combination therein.

In a related embodiment, the implant comprises hygroscopic properties allowing for cellular and/or chemical diffusion and/or communication between internal bioceramic-containing biomaterials and an external implant surface.

In a related embodiment, the implant is process-strengthened utilizing strain hardening, compression annealing, cross-linking, or addition of strengthening additive.

In a related embodiment, the implant includes variable zones of differing bioceramic-containing solid content to impart regional mechanical and biological functions.

In a related embodiment, the implant includes variable zones of differing thermoplastic physical or chemical properties that, in combination with the bioceramic-containing solid, imparts regional zones having different mechanical and biological functions within the implant.

In another embodiment, a bioceramic-containing biomaterial-derived thermoplastic filament is provided. The bioceramic-containing biomaterial-derived thermoplastic filament includes a bioceramic-containing component combined with a thermoplastic resin to form a mixture such that there is even dispersal of the bioceramic-containing component in the thermoplastic resin. The thermoplastic resin includes nylon, acrylonitrile butadiene styrene (ABS), polycarbonate, polyetherimide, polycaprolactone, polymethylmethacrylate (PMMA), acrylic, or polyacryletherketones, and the bioceramic-containing component is in a form of a powder, granule, or fiber. The mixture is molded or extruded into a filament or pellet. The filament or pellet contains a minimum of 0.1% bioceramic-containing material by weight. The bioceramic-containing component has a diameter no greater than 70% of the filament or pellet diameter. The filament is substantially flexible and configured to be rolled onto a spool. The filament is adapted for the manufacture of medical devices using additive manufacturing methods. The filament or pellet has undergone a terminal sterilization and packaging process via irradiation, heat, or chemical treatment.

In another embodiment, a filament adapted for use in a volumetric or 3D printer or mold is provided. The filament includes a thermoplastic of a first predetermined quantity and a processed bioceramic-containing material of a second predetermined quantity. The first and second predetermined quantities are selected to define a desired ratio of bioceramic-containing material to thermoplastic to modulate physical or biological properties in an implant manufactured using the filament.

In a related embodiment, the bioceramic-containing material is distributed substantially evenly with the thermoplastic in predetermined areas of the filament.

In a related embodiment, the bioceramic-containing material is distributed substantially evenly with the thermoplastic substantially throughout the filament.

In a related embodiment, the bioceramic-containing material has a particle size of less than 1,000 μm.

In a related embodiment, a mass of the thermoplastic is at least 1.5 times the mass of the bioceramic-containing material in the filament.

In a related embodiment, the bioceramic-containing material comprises at least one of calcium phosphate, tricalcium phosphate, hydroxyapatite, multiphasic calcium phosphate, calcium silicate, sodium silicate, or silicate-substituted calcium phosphate.

In a related embodiment, the bioceramic-containing material is in a powdered form, a granular form, an elongated form, or a fiber form, wherein the powder form and the granular forms have particles less than 1,000 μm in size.

In a related embodiment, the bioceramic-containing material is mixed with the thermoplastic in a ratio, the ratio is determined by mass, wherein a mass of the thermoplastic is from 2 to 100 times a mass of the bioceramic-containing material.

In a related embodiment, the bioceramic-containing material is mixed with the thermoplastic in a specific ratio, the ratio is determined by mass, wherein a mass of the thermoplastic is from 10 to 50 times a mass of the bioceramic-containing material.

In a related embodiment, the thermoplastic includes at least one of nylon, acrylonitrile butadiene styrene (ABS), polycarbonate, polyetherimide, polycaprolactone, polymethylmethacrylate (PMMA), acrylic, or polyacryletherketones.

In a related embodiment, the filament contains a minimum of 0.1% bioceramic-containing material by weight.

In a related embodiment, the bioceramic-containing material includes at least one of calcium phosphate, tricalcium phosphate, hydroxyapatite, multiphasic calcium phosphate, calcium silicate, sodium silicate, and/or silicate-substituted calcium phosphate, and the bioceramic-containing material is a granule or a fiber.

In another embodiment, a surgical implant manufactured from a thermoplastic extrusion is provided.

In a related embodiment, the surgical implant is manufactured utilizing volumetric printing, injection molding, machining, sintering, or forming.

In a related embodiment, the surgical implant includes hygroscopic properties allowing for cellular and/or chemical diffusion.

In a related embodiment, the surgical implant is process-strengthened utilizing strain hardening, compression annealing, cross-linking, or addition of strengthening additive, in order to accommodate physiological loading without failure.

### BRIEF DESCRIPTION OF THE DRAWINGS

No figures accompany this filing.

### DETAILED DESCRIPTION

All ranges or values of properties of the embodiments described herein include the endpoints of the ranges specified.

A biomaterial filament is provided. In an embodiment, the biomaterial filament includes a thermoplastic polymer and a bioceramic-containing component.

The thermoplastic polymer is a biocompatible thermoplastic configured to be safely introduced to a surface of a human bone. In some examples, the thermoplastic polymer is selected from a group consisting of nylon, acrylonitrile butadiene styrene (ABS), polycarbonate, acrylic, polyaryletherketones, polymethyl methacrylate, polycaprolactone, polyetherimide, and combinations thereof. In some examples, the thermoplastic polymer is processed prior to being introduced to the bioceramic-containing component. The processing of the thermoplastic polymer may include crushing or pulverizing the thermoplastic polymer into a powder or granulate. In a preferred example, the thermoplastic polymer has a particle size of 1,000 microns or less to facilitate effective combination with the bioceramic-containing component.

In an example, the thermoplastic polymer is a polymethyl methacrylate (PMMA) formulation. Particularly, the PMMA formulation may be a formulation that meets biocompatibility requirements set forth in ISO 10993, and meets property requirements set forth in ASTM 3087-15 Standard Specification for Acrylic Molding Resins for Medical Implant Applications. In some examples, the PMMA formulation includes a material density of between 1.17 g/cm³ and 1.20 g/cm³. In some examples, the PMMA formulation includes a residual monomer content of a maximum of 0.5% by weight of the final PMMA produced.

In some examples, the PMMA formulation include other parameters within a range suitable for safe implantation in a human, and is accordingly considered to be biocompatible, and more specifically, biocompatible in humans. For example, the PMMA formulation may include a weight average molecular weight (M_{w}) of between 80,000 and 200,000 Daltons and/or a number average molecular weight (Mₙ) of between 40,000 and 80,000. Alternatively or in addition, the PMMA formulation may include a polydispersity index (PDI) of between 1.0 and 2.0. Alternatively or in addition, the PMMA formulation may include a melt flow rate of between 0.5 g/10 min and 20.0 g/10 min.

Alternatively or in addition, the PMMA resulting from the formulation described herein may exhibit mechanical properties within specified ranges. For example, the resulting PMMA may exhibit tensile elongation at break of between 1.0% and 30.0%. Alternatively or in addition, the PMMA may exhibit a tensile modulus of elasticity of between 1.0 GPa and 10.0 GPa. Alternatively or in addition, the PMMA may exhibit tensile strength of between 20 MPa and 90 MPa.

The PMMA resulting from the formulation described herein may be a thermoplastic and is extrudable at least at temperatures between 160-250° C., preferably the PMMA is extrudable at a temperature of between 200° C. and 250° C. In an example, the PMMA may be heated into a liquified material. The liquified material may be extruded into a filament that is flexible and configured to allow for spooling. Alternatively or in addition, the PMMA resulting from the formulation described herein may be processed into a powder, granulate, or pellet.

In some examples, the PMMA material can be terminally sterilized via standard means for biologics and medical devices, for example by irradiation or chemical treatment.

In some examples, the PMMA material can be used to manufacture a medical device utilizing volumetric printing, injection molding, machining, sintering, forming or similar means.

In some examples, the PMMA material may be formed into a filament form and can be used to produce devices using Fused Deposition Modeling (FDM) or Fused Filament Fabrication (FFF) methods (commonly called 3D printing). Alternatively or in addition, the PMMA material may be formed into a powder and may be used to produce devices using Selective Laser Sintering (SLS) methods.

The PMMA is treated such that the PMMA has properties suitable for use in human patients. For example, methyl methacrylate, toluene and azobisisobutyronitrile are combined in a reaction flask and degassed with nitrogen. The contents contained in the reaction flask are then heated to 70° C. for 24 hours, in which a polymerization reaction occurs. The polymerization reaction is quenched by cooling the solution to room temperature, and the PMMA is precipitated out by pouring the polymer solution into heptane. The resulting polymer powder is washed with heptane and methanol, and then subsequently dried in a vacuum oven. The resulting polymer powder meets biocompatibility requirements set forth in ISO 10993, and meets property requirements set forth in ASTM 3087-15 Standard Specification for Acrylic Molding Resins for Medical Implant Applications.

The biomaterial filament further includes a bioceramic-containing component. Bioceramics are biocompatible, bioactive materials used for repairing or replacing damaged bone. These biomaterials support new bone growth, and may interact with bone tissue when implanted to be totally integrated in several stages and eventually replaced by the newly formed bone. In some examples, the bioceramic-containing component is a synthetic biomaterial that may primarily be composed of calcium phosphate, calcium silicate, sodium silicate, or silicate-substituted calcium phosphate. As mentioned, the bioceramic-containing component is synthetic, and accordingly, does not include natural bone from one or more human tissue donors or one or more animal carcasses. Rather, the bioceramic-containing component may be formed by blending a calcium source and mineral. In an example, the calcium source is tricalcium phosphate, and the mineral is hydroxyapatite. In an example, the bioceramic-containing component is a blend of tricalcium phosphate and hydroxyapatite. In another example, the bioceramic-containing component is a blend of 80 wt % tricalcium phosphate and 20% hydroxyapatite. In another example the bioceramic-containing material includes a silicate component, and may be blended with calcium, sodium, or phosphate. In some examples, the bioceramic-containing component is processed prior to being introduced to the thermoplastic polymer. The processing of the bioceramic-containing component may include crushing or pulverizing the bioceramic-containing component into a powder or granulate. In some examples, the bioceramic-containing component powder or granulate has a particle size of between 125 μm and 250 μm. This particle size is particularly ideal for extrusion and volumetric printing applications. To be acceptable for medical use, such bioceramic-containing materials are manufactured under certified quality management systems such as ISO 9001 and/or ISO 13485 to ensure consistent product safety and efficacy.

The biomaterial filament is formed by combining the thermoplastic polymer and the bioceramic-containing component by a gravimetric process, and may include a specific ratio of thermoplastic polymer:bioceramic-containing component. In some examples, the mass of the thermoplastic polymer and the mass of the bioceramic-containing component is combined in a ratio of between 2:1 by weight and 50:1 by weight. In an example, the mass of the thermoplastic polymer and the mass of the bioceramic-containing component is combined in a ratio of 2:1 by weight. In another example, the mass of the thermoplastic polymer and the mass of the bioceramic-containing component is combined in a ratio of 10:1 by weight. In an example, the mass of the thermoplastic polymer and the mass of the bioceramic-containing component is combined in a ratio of 50:1 by weight. In an example, the bioceramic-containing component and the thermoplastic polymer are placed into separate gravimetric feeders on an extrusion system. In an example, the thermoplastic polymer is heated between a glass transition temperature of the thermoplastic polymer and a melting temperature of the thermoplastic polymer prior to combining the thermoplastic polymer with the bioceramic-containing component.

As described above, forming the biomaterial filament includes combining the thermoplastic polymer and the bioceramic-containing component. The combining of the thermoplastic polymer and the bioceramic-containing component may occur by mixing the thermoplastic polymer and the bioceramic-containing component within an extrusion chamber to form a mixture, and heating the mixture to a temperature sufficient to melt the mixture into a flowable state. In an example, the mixture is heated to a temperature between 160° C. and 250° C. In some examples, the bioceramic-containing component is provided in a powdered or granular form having particles equal to or less than 500 μm in size. In an example, the mixture is formed by mixing the thermoplastic polymer and the bioceramic-containing component in a single or twin auger screw apparatus at a speed sufficient for making an extruded biomaterial filament of a desired diameter. In some examples, mixing the bioceramic-containing component with the thermoplastic resin occurs within an extrusion chamber subjected to heat and/or pressure by an auger screw, and the auger screw are configured to disperse the bioceramic-containing solid in the at least one thermoplastic resin. In some embodiments, the mixing further includes physical agitation, electrostatic adhesion, or ultrasonic agitation to create uniform mixing of the bioceramic-containing component and the thermoplastic resin. In some examples, the bioceramic-containing component is mixed with the at least one thermoplastic resin in a liquid state, undergoing mechanical agitation prior to or during the extrusion process. In some examples, mixing the bioceramic-containing component with a thermoplastic liquid to create a uniform dispersal prior to being placed in an extrusion chamber occurs, and the mixing includes impeller agitation or ultrasonic agitation resulting in a heated liquid state, the mixed bioceramic-containing solid and thermoplastic liquid having a temperature above the melting point of the thermoplastic liquid, wherein the bioceramic-containing solid is added during and/or prior to the agitation and/or heating. In an example, the bioceramic-containing component is mixed with the thermoplastic resin in a predetermined ratio, the ratio is determined by mass, wherein the mass of the thermoplastic resin is from 10 to 50 times the mass of the bioceramic-containing solid. In an example, the bioceramic-containing component is mixed with the thermoplastic in a ratio, the ratio is determined by mass, wherein a mass of the thermoplastic is from 2 to 100 times a mass of the bioceramic-containing component.

In some examples, the diameter of the biomaterial filament is between 1.5 mm and 3.0 mm. In another example, the diameter of the biomaterial filament is between 1.6 mm and 1.8 mm. In another example, the diameter of the biomaterial filament is between 2.5 mm and 2.9 mm. The resulting biomaterial filament is flexible to allow for spooling. In some examples, the biomaterial filament is sterilized by standard means for biologics and medical devices. In some examples, the sterilization of the biomaterial filament is carried out by irradiation or chemical exposure. The biomaterial filament can be terminally sterilized to a Safety Assurance Level (SAL) of 10⁻⁶. In some examples, the biomaterial filament is substantially flexible such that it is configured to roll onto a spool. In some examples, the filament contains a minimum of 0.1% bioceramic-containing material by weight. In an example, a mass of the thermoplastic is at least 1.5 times the mass of the bioceramic-containing component in the filament.

A method for producing a biomaterial filament is also provided. The biomaterial filament may be produced by extrusion of the thermoplastic polymer and the bioceramic-containing component. The biomaterial filament may be formed into various desired particular shapes by volumetric printing, injection molding, machining, sintering, or by similar processes. In an example, the biomaterial filament may be formed to desired specific shapes by 3D printing methods. These 3D printing methods include, but are not necessarily limited to, Fused Deposition Modeling (FDM) or Fused Filament Fabrication (FFF) methods. In examples where the biomaterial filament is particularly formed to desired shapes, the biomaterial filament is loaded into a printing device, and the printing device heats the biomaterial filament above the melting temperature of the biomaterial filament to create a flowable mixture. In an example, the device heats the biomaterial filament to between 245° C. and 255° C. to liquefy the biomaterial filament. The device then prints the liquified material in consecutive layers, and the liquified material solidifies after printing to form a biomaterial graft. The biomaterial graft may be shaped as desired at least due to it being configured to be formed by the 3D printing methods described above.

The device for printing the biomaterial filament to form may be configured to operate and/or actually operate with particular parameters. These parameters may influence the quality of the resulting biomaterial graft. In some examples, the device includes a nozzle having a diameter of about or exactly 0.4 mm or 0.8 mm, and the nozzle is used to dispense the liquified biomaterial filament to form the biomaterial graft. In another example, the device is configured to operate and/or actually operates at a print speed of between 15 mm/s and 30 mm/s, wherein liquified biomaterial filament is configured to be dispensed and/or is actually dispensed from the nozzle as the nozzle moves over the build surface at the specified speed. In another example, the device is configured to dispense and/or actually dispenses layers of liquified biomaterial filament to form the biomaterial graft. The layers may have a height between 0.1 mm and 0.4 mm. In another example, the infill density or print density of the biomaterial graft is between 50% and 100%. In another example, the liquified biomaterial filament is dispensed from the device to form the biomaterial graft. The build surface is at a temperature of about 100° C. at the time of dispensing of the biomaterial filament, and the graft surface temperature is gradually reduced to about 40° C. The cooling of the biomaterial graft may occur with the assistance of a fan in some examples. Alternatively, the cooling of the biomaterial graft may occur without the assistance of a fan.

In addition, in some examples, a plurality of materials can be simultaneously used to print a biomaterial graft. For example, a biomaterial filament may be loaded into a device having more than one print heads, including, for example, a first print head and a second print head. The biomaterial filament may be loaded into the device to be dispensed from the first print head. In addition, a second material may be loaded into the device to be dispensed from the second print head. In some examples, the second material may be a support material, pure thermoplastic, or a second biomaterial filament having a different or the same weight ratio of thermoplastic polymer-to-bioceramic containing component as the biomaterial filament loaded into the device to be dispensed from the first print head.

The device prints a biomaterial graft using the biomaterial filament loaded therein. The resulting biomaterial graft is a medical device and is a bioactive osteoconductive surgical implant that supports bone growth. In some examples, the implant includes hygroscopic properties allowing for cellular and/or chemical diffusion and/or communication between internal bioceramic-containing biomaterials and an external implant surface. The bioactive osteoconductive attributes of the biomaterial graft are present in the biomaterial graft at least because the thermoplastic polymer has been incorporated with the bioceramic-containing component. In some examples, bioceramic-containing component is uniformly dispersed within the biomaterial graft. Alternatively, the bioceramic-containing component is non-uniformly distributed in the biomaterial graft, for example, by being strategically located in areas where bioactivity is desired. For example, for a biomaterial graft intended for use as a spinal fusion implant, the bioceramic-containing component is concentrated on the biomaterial graft's superior and inferior surfaces to interact with adjacent vertebral bodies once implanted in a human patient. The localized areas of the bioceramic-containing component aid in directing a desired biologic response once the biomaterial graft is implanted in a human patient.

In some examples, the bioceramic-containing component may be exposed on the biomaterial graft surface or surfaces to enhance osteoconductive properties compared to biomaterial grafts without bioceramic-containing components exposed on the biomaterial graft surface. In some examples, the bioceramic-containing component may be exposed by mechanical methods such as abrasive sanding. Alternatively or in addition, the bioceramic-containing component may be exposed on a surface or surfaces of the biomaterial graft by contacting the biomaterial graft with one or more solvents or one or more solutions to remove a portion of the thermoplastic polymer while retaining the bioceramic-containing component on the biomaterial graft surface. In some examples, the implant includes hygroscopic properties allowing for cellular and/or chemical diffusion and/or communication between internal bioceramic-containing biomaterials and an external implant surface.

The bioceramic-containing component may absorb fluid from its surroundings, and accordingly, the biomaterial graft possesses hygroscopic properties. In some examples, the absorbed fluid may contain nutrients and/or cells that facilitate a healing response.

The biomaterial graft further possesses biomechanical properties appropriate for its intended use and can accommodate relevant physiological loading without failure. For example, the biomaterial graft is further processed after printing, such as by utilizing strain hardening, compression annealing, cross-linking, addition of strengthening additive, or similar means to bolster the biomaterial graft's biomechanical properties. Furthermore, the bioceramic-containing component content influences biomaterial properties in a controlled manner. For example, the biomaterial graft may possess regions of lower bioceramic-containing component concentration to emphasize the mechanical attributes of the thermoplastic polymer. Alternatively or in addition, the biomaterial graft may possess regions of higher bioceramic-containing component concentration to impart more bone-like mechanical qualities. Furthermore, the implant may include variable zones of differing bioceramic-containing solid content to impart regional mechanical and biological functions. Alternatively or in addition, the implant includes variable zones of differing thermoplastic physical or chemical properties that, in combination with the bioceramic-containing solid, imparts regional zones having different mechanical and biological functions within the implant.

The biomaterial graft described herein has advantages over previously developed biomaterial grafts. A non-exhaustive list of advantages is described. For example, the biomaterial graft described herein is an osteoconductive biomaterial that can elicit a biological response to support bone growth. The bioceramic-containing component is integrated throughout the biomaterial filament rather than being strictly surface-coated onto the biomaterial filament or biomaterial graft. Biomaterial filaments and/or biomaterial grafts having bioceramic-containing components only surface-coated onto the biomaterial filament or biomaterial grafts are susceptible to flaking and/or peeling of bioceramic-containing components, and accordingly losing their bone-like properties. The biomaterial filament and biomaterial graft described herein possesses sufficient material and mechanical properties such that it can be used to fabricate physiologic load bearing devices/implants. The biomaterial filament and biomaterial graft described herein is radiolucent for visualization with common clinical imaging methods. The biomaterial filament and biomaterial graft can be used with 3D printing manufacturing methods to create medical devices/implants.

Furthermore, the biomaterial filament is not necessarily limited to being a filament, per se. Rather, the biomaterial filament or bioceramic-containing biomaterial-derived thermoplastic extrusion may be produced in alternate forms such as pellet, bar, molding, or other 3D printing material stock. The biomaterial filament may be used in other manufacturing methods such as injection molding, traditional machining, sintering, or forming methods other than 3D printing methods as well.

What is claimed is:

1. A method of generating a bioceramic-containing biomaterial-derived thermoplastic extrusion, the method comprising:
combining a bioceramic-containing solid with at least one thermoplastic resin, wherein the bioceramic-containing solid is uniformly dispersed in the resin; and
extruding the combined bioceramic-containing solid and the at least one thermoplastic resin to form an extrusion and to create a net shape,
wherein the net shape is selected from a group consisting of a filament, a pellet, a bar, a molding, and a three-dimensional printing material stock.

2. The method of claim 1, further comprising:
mixing the bioceramic-containing solid with a thermoplastic pellet in a solid state prior to or during extruding the bioceramic-containing solid and the at least one thermoplastic resin,
wherein mixing the bioceramic-containing solid with the thermoplastic pellet occurs below a glass transition temperature of the thermoplastic pellet, and
the mixing further comprises physical agitation, electrostatic adhesion, or ultrasonic agitation to create uniform mixing of the bioceramic-containing solid and the thermoplastic resin.

3. The method of claim 2, wherein mixing the bioceramic-containing solid with the at least one thermoplastic resin occurs within an extrusion chamber subjected to heat and/or pressure by an auger screw, the auger screw configured to disperse the bioceramic-containing solid in the at least one thermoplastic resin.

4. The method of claim 1, wherein the bioceramic-containing solid is mixed with the at least one thermoplastic resin in a liquid state, undergoing mechanical agitation prior to or during the extrusion process.

5. The method of claim 4, further comprising:
mixing the bioceramic-containing solid with a thermoplastic liquid to create a uniform dispersal prior to being placed in an extrusion chamber; and
the mixing comprising impeller agitation or ultrasonic agitation resulting in a heated liquid state, the mixed bioceramic-containing solid and thermoplastic liquid having a temperature above the melting point of the thermoplastic liquid, wherein the bioceramic-containing solid is added during and/or prior to the agitation and/or heating.

6. The method of claim 1, wherein the bioceramic-containing solid comprises at least one of calcium phosphate, tricalcium phosphate, hydroxyapatite, multiphasic calcium phosphate, calcium silicate, sodium silicate, or silicate-substituted calcium phosphate.

7. The method of claim 1, wherein the bioceramic-containing solid is provided in a powdered or granular form having particles equal to or less than 500 μm in size.

8. The method of claim 1, wherein the bioceramic-containing solid is mixed with the thermoplastic resin in a predetermined ratio, the ratio is determined by mass, wherein the mass of the thermoplastic resin is from 10 to 50 times the mass of the bioceramic-containing solid.

9. The method of claim 1, wherein the filament is configured to roll onto a spool.

10. The method of claim 1, wherein the extrusion undergoes terminal sterilization via an irradiation, heat, or chemical treatment.

11. A bioceramic-containing biomaterial-derived thermoplastic extrusion comprising:
a solid derived from bioceramic-containing biomaterial, the bioceramic-containing biomaterial uniformly dispersed in a thermoplastic resin,
wherein the bioceramic-containing biomaterial-derived thermoplastic extrusion is shaped as a filament, pellet, bar, molding, or three-dimensional printing material.

12. The extrusion of claim 11, wherein the bioceramic-containing biomaterial comprises at least one of calcium phosphate, tricalcium phosphate, hydroxyapatite, multiphasic calcium phosphate, calcium silicate, sodium silicate, or silicate-substituted calcium phosphate.

13. The extrusion of claim 11, wherein the thermoplastic resin comprises nylon, ABS, polycarbonate, acrylic, polyaryletherketones, polymethyl methacrylate, polycaprolactone, or polyetherimide.

14. The extrusion of claim 11, further comprising a minimum of 0.1% bioceramic-containing biomaterial by weight.

15. The extrusion of claim 11, wherein the extrusion is formed into a filament, the filament being substantially flexible, such that the filament is configured to be rolled onto a spool.

16. The extrusion of claim 11, wherein the extrusion undergoes terminal sterilization via an irradiation, heat, or chemical treatment.

17. An osteoconductive surgical implant comprising:
a bioceramic-containing biomaterial-derived thermoplastic extrusion,
wherein the surgical implant incorporates a combination of a bioceramic-containing solid and a thermoplastic with dispersal of the bioceramic-containing solid in the thermoplastic.

18. The surgical implant of claim 17, manufactured utilizing additive manufacturing, volumetric printing, injection molding, machining, sintering, or forming.

19. The surgical implant of claim 17, wherein the dispersal of the bioceramic-containing solid within the thermoplastic is uniform.

20. The surgical implant of claim 17, wherein at least a portion of the bioceramic-containing solid is exposed at a surface of the implant, and the exposed bioceramic-containing solid expresses osteoconductive properties and imparting the properties to the implant.

21. The surgical implant of claim 20, wherein the bioceramic-containing solid is mechanically or chemically exposed on the surface in a controlled manner for exposure of osteoconductive elements where biologic response is desired, wherein
the chemical exposure comprises treatment of the implant with an acid, ethanol, or a combination therein.

22. The surgical implant of claim 17, wherein the implant comprises hygroscopic properties allowing for cellular and/or chemical diffusion and/or communication between internal bioceramic-containing biomaterials and an external implant surface.

23. The surgical implant of claim 17, wherein the implant is process-strengthened utilizing strain hardening, compression annealing, cross-linking, or addition of strengthening additive.

24. The surgical implant of claim 17, wherein the implant includes variable zones of differing bioceramic-containing solid content to impart regional mechanical and biological functions.

25. The surgical implant of claim 17, wherein the implant includes variable zones of differing thermoplastic physical or chemical properties that, in combination with the bioceramic-containing solid, imparts regional zones having different mechanical and biological functions within the implant.

26. A bioceramic-containing biomaterial-derived thermoplastic filament comprising:
a bioceramic-containing component combined with a thermoplastic resin to form a mixture such that there is even dispersal of the bioceramic-containing component in the thermoplastic resin;
wherein the thermoplastic resin comprises nylon, acrylonitrile butadiene styrene (ABS), polycarbonate, polyetherimide, polycaprolactone, polymethylmethacrylate (PMMA), acrylic, or polyacryletherketones, and the bioceramic-containing component is in a form of a powder, granule, or fiber,
the mixture being molded or extruded into a filament or pellet;
the filament or pellet containing a minimum of 0.1% bioceramic-containing material by weight;
the bioceramic-containing component having a diameter no greater than 70% of the filament or pellet diameter;
the filament being substantially flexible and configured to be rolled onto a spool;
the filament adapted for the manufacture of medical devices using additive manufacturing methods; and
the filament or pellet having undergone a terminal sterilization and packaging process via irradiation, heat, or chemical treatment.

27. A filament adapted for use in a volumetric or 3D printer or mold, the filament comprising:
a thermoplastic of a first predetermined quantity; and
a processed bioceramic-containing material of a second predetermined quantity;
the first and second predetermined quantities being selected to define a desired ratio of bioceramic-containing material to thermoplastic to modulate physical or biological properties in an implant manufactured using the filament.

28. The filament of claim 27, wherein the bioceramic-containing material is distributed substantially evenly with the thermoplastic in predetermined areas of the filament.

29. The filament of claim 27, wherein the bioceramic-containing material is distributed substantially evenly with the thermoplastic substantially throughout the filament.

30. The filament of claim 27, wherein the bioceramic-containing material has a particle size of less than 1,000 μm.

31. The filament of claim 27, wherein a mass of the thermoplastic is at least 1.5 times the mass of the bioceramic-containing material in the filament.

32. The filament of claim 27, wherein the bioceramic-containing material comprises at least one of calcium phosphate, tricalcium phosphate, hydroxyapatite, multiphasic calcium phosphate, calcium silicate, sodium silicate, or silicate-substituted calcium phosphate.

33. The filament of claim 27, wherein the bioceramic-containing material is in a powdered form, a granular form, an elongated form, or a fiber form, wherein the powder form and the granular forms have particles less than 1,000 μm in size.

34. The filament of claim 27, wherein the bioceramic-containing material is mixed with the thermoplastic in a ratio, the ratio is determined by mass, wherein a mass of the thermoplastic is from 2 to 100 times a mass of the bioceramic-containing material.

35. The filament of claim 27, wherein the bioceramic-containing material is mixed with the thermoplastic in a specific ratio, the ratio is determined by mass, wherein a mass of the thermoplastic is from 10 to 50 times a mass of the bioceramic-containing material.

36. The filament of claim 27, wherein the thermoplastic comprises at least one of nylon, acrylonitrile butadiene styrene (ABS), polycarbonate, polyetherimide, polycaprolactone, polymethylmethacrylate (PMMA), acrylic, or polyacryletherketones.

37. The filament of claim 27, wherein the filament contains a minimum of 0.1% bioceramic-containing material by weight.

38. The filament of claim 27, wherein the bioceramic-containing material comprises at least one of calcium phosphate, tricalcium phosphate, hydroxyapatite, multiphasic calcium phosphate, calcium silicate, sodium silicate, and/or silicate-substituted calcium phosphate, and the bioceramic-containing material is a granule or a fiber.

39. A surgical implant manufactured from a thermoplastic extrusion.

40. The surgical implant of claim 39 manufactured utilizing volumetric printing, injection molding, machining, sintering, or forming.

41. The surgical implant of claim 39, wherein the surgical implant comprises hygroscopic properties allowing for cellular and/or chemical diffusion.

42. The surgical implant of claim 39, wherein the surgical implant is process-strengthened utilizing strain hardening, compression annealing, cross-linking, or addition of strengthening additive, in order to accommodate physiological loading without failure.
